(19) United States
(12) Patent Application Publication
(10) Pub. No.: US 2022/0332985 A1
(43) Pub. Date: Oct. 20, 2022

(54) TACKIFIED HOTMELT ADHESIVE

(71) Applicant: The Procter & Gamble Company, Cincinnati, OH (US)

(72) Inventors: Torsten LINDNER, Kronberg (DE); Matthias MORAND, Sulzbach (DE); Robert Haines TURNER, Cincinnati, OH (US); Filipa Alexandra Macedo Tavares COELHO, Frankfurt am Main (DE)

(21) Appl. No.: 17/684,457

(22) Filed: Mar. 2, 2022

Related U.S. Application Data
(60) Provisional application No. 63/176,921, filed on Apr. 20, 2021.

(51) Int. Cl. C09J 123/16; C09J 11/06

(52) U.S. Cl. CPC C09J 123/16; C09J 11/06; C09J 2203/358; C09J 2301/304

(57) ABSTRACT

An hotmelt adhesive for absorbent article (20), wherein the hotmelt adhesive comprises at least one low molecular weight metallocene-catalyzed polyolefin, at least one high molecular weight polyolefin having a peak molecular weight of from 130,000 g/mol to 700,000 g/mol and at least one tackifier. The hotmelt adhesive comprises less than 10% by weight of mineral oil. The hotmelt adhesive is particularly useful to form a core wrap bond (82, 84, 86) and/or a patch-to-core bond (78).

### CROSS REFERENCE TO RELATED APPLICATION

This application claims the benefit, under 35 U.S.C. § 119(e), of U.S. Provisional Patent Application Ser. No. 63/176,921, filed on Apr. 20, 2021, the entire disclosure of which is incorporated herein by reference.

### TECHNICAL FIELD

The present disclosure is directed at a hotmelt adhesive. The hotmelt adhesive comprises two polyolefins having different peak molecular weight and a tackifier. The hotmelt adhesive may be used in for personal hygiene absorbent articles such as diapers or adult incontinence products. The hotmelt adhesive is particularly useful to form core wrap bonds and patch-to-core bond in absorbent articles.

### BACKGROUND

Disposable absorbent articles, such as diapers, training pants or adult incontinence articles, comprise various components that are bonded together directly or indirectly. Hotmelt adhesives have been used to bond individual layers, in particular topsheet, backsheet and absorbent core, which together form the chassis of the article. Hotmelt adhesives have also been used to bond other discrete components, such as fasteners and leg elastics or cuffs, to the chassis of the article. The hotmelt adhesives are often called construction adhesives for these applications as they help constructing the absorbent article from individual components. Other bonding means such as fusion bonding and ultrasonic bonding are typically not practical for thin nonwoven layers and when large surface are to be bonded.

Hotmelt adhesives are solid at room temperature, and are thus applied heated in the molten state by contact or non-contact nozzles, as is known in the art. Hotmelt adhesives are made by combining one or more backbone polymers with additive components in a substantially uniform thermoplastic blend. Typical additive components include tackifiers, plasticizers, and/or waxes. Plasticizers such as mineral oil allows the hotmelt to be applied at lower temperature by reducing the viscosity of the composition. Various hotmelt adhesives have been disclosed in the art. There has been a recent effort to develop new hotmelt adhesives reducing or eliminating plasticizer or tackifier in the hotmelt adhesives, especially in the field of hygiene article. One difficulty is that the various components of absorbent articles have different geometries, surface properties and heat resistance.

WO2019/204,541 (Turner et al.) discloses that adhesive compositions having a viscosity from about 2.000 mPa.s to about 11,500 mPa.s at 150° C., a Storage Modulus (G′) at 37° C. between about 3 MPa to about 9.5 MPa, and a Yield Stress at 37° C. of from about 0.8 MPa to about 1.45 Mpa perform well in adhesive stiff film laminates and typically also perform well in nonwoven-nonwoven laminates. These hotmelt adhesives comprise a copolymer and preferably have a Toughness at 37° C. of from about 2 MJ/m³ to about 8 MJ/m³.

Pure polyolefins manufactured by metallocene catalysts (mPO) have been suggested for use as hotmelt adhesives. mPO can be produced without impurities and have nearly no odor. They are also accessible from renewable substances (e.g., via ethylene from natural ethanol) and can be rendered biodegradable. They have a low density which enables lower coating weights (less mass per volume) and they can be produced at low cost (polymerization in one step, from low cost monomers). mPO are also lotion resistant and bonds formed from pure mPO do not age over time as they do not release low molecular weight ingredients that diffuse into other parts of the diaper.

Hotmelt compositions comprising metallocene-catalyzed propylene-based copolymers have been proposed, see for example WO2016/153,663A1, WO2014/194,074A1 and US2020/0108,168A1. US2016/053,149A1 (Clamant) for example discloses a ready-to-use hotmelt adhesive comprising at least 95% of one or more polyolefin copolymer waxes, which have been prepared by means of metallocene catalysts, characterized in that the polyolefin copolymer wax consists of propylene and one or more further monomers selected from ethylene and branched or unbranched 1-alkenes having 4 to 20 C atoms and the content of structural units derived from propylene in the copolymer waxes amounts to 80 to 99.9% by weight, and the hotmelt adhesive has a surface tension of the melt, measured at a temperature of 170° C., of at most 23 mN/m.

Hotmelt compositions consisting of pure mPO's however were found to have some limitations. In particular, the open time of mPO based hotmelt adhesives is comparably short, and this requires for some applications the additional implementation of a bonding roll for the bonding to take place process, which goes along with capital cost.

When developing a blend based on mPO, the benefits of the pure mPO should be impacted as little as possible while the above indicated limitations need to be overcome. The hotmelt adhesive compositions should be easily applicable by slot coating or spraying.

Conventionally, mineral oils are added to hotmelt adhesives to compensate for a too high hotmelt blend viscosity. Mineral oils have several drawbacks such as being volatile (odor), diffusing into other substrate like PE films or onto the surfaces of other materials (like SAP) over time, which weakens the bond and deteriorates the function of other parts of the diaper. Mineral oils also contribute to lower thermal stability of the adhesive in the heated melting tank during processing causing faster thermal degradation of the adhesive over time.

There is a continuous need for hotmelt adhesives that comprise little or no mineral oil and that high performing for selected uses in absorbent articles.

### SUMMARY OF THE INVENTION

In a first aspect, the invention is for an hotmelt adhesive comprising:
at least one low molecular weight metallocene-catalyzed polyolefin having a peak molecular weight below 130,000 g/mol, wherein the peak molecular weight is measured according to the Peak Molecular Weight (Mp) Measurement Method disclosed herein;
at least one high molecular weight polyolefin having a peak molecular weight of from 130,000 g/mol to 700,000 g/mol; and
at least one tackifier.

The hotmelt adhesives according to the invention may be formulated with low amount (less than 10% by weight) and are preferably free of mineral oil.

In a second aspect the invention is for an absorbent article comprising adhesive bonds formed by the hotmelt adhesive. The hotmelt adhesive was found to be particularly useful to make core wrap bonds, patch-to-core bonds.

The above-mentioned and other features and advantages of the present disclosure, and the manner of attaining them, will become more apparent and the disclosure itself will be better understood by reference to the following description.

### BRIEF DESCRIPTION OF THE DRAWINGS

FIG. 1 shows a perspective view of an exemplary taped diaper in a closed configuration as it would be when worn by a wearer;

FIG. 2 shows a simplified view of the garment-facing side of the diaper of FIG. 1, with the diaper being flattened out;

FIG. 3 shows a simplified view of the wearer-facing side of the diaper of FIG. 1, with the diaper flattened out;

FIG. 4 shows a top view of an exemplary absorbent core with the top layer partially removed;

FIG. 5 shows a longitudinal cross-section view of the absorbent core of FIG. 4;

FIG. 6 shows transversal cross-section view of the absorbent core of FIG. 4;

FIG. 7 shows a cross-section of an exemplary diaper.

### DETAILED DESCRIPTION

The following term explanations may be useful in understanding the present disclosure.

“Absorbent article” or “personal hygiene absorbent article”, as used herein, refers to devices that are placed against or in proximity to the body of the wearer to absorb and contain the various exudates discharged from the body. Absorbent articles of the invention include taped diapers and pant diapers. Their size may be adapted for babies, young children or for adults suffering incontinence. While the absorbent articles are disposable and typically discarded after usage, they are preferably recycled or otherwise disposed of in an environmentally compatible manner.

The terms “elastic,” “elastomer,” and “elastomeric” refer to a material which generally is able to extend to a strain of at least 50% without breaking or rupturing, and is able to recover substantially to its original dimensions after the deforming force has been removed.

“Longitudinal” refers to the direction along the axis (80 in the Figures) extending from the midpoint of the front waist edge to the midpoint of the rear waist edge of the absorbent article, and that bisects the absorbent article into a left half and right half. “Transversal ” refers to the direction perpendicular to the longitudinal line (90 in the Figures).

As used herein, the terms “nonwoven”, nonwoven layer” or “nonwoven web” are used interchangeably to mean an engineered fibrous assembly, primarily planar, which has been given a designed level of structural integrity by physical and/or chemical means, excluding weaving, knitting or papermaking (ISO 9092:2019 definition). The directionally or randomly orientated fibers, are bonded by friction, and/or cohesion and/or adhesion. The fibers may be of natural or synthetic origin and may be staple or continuous filaments or be formed in situ. Commercially available fibers have diameters ranging from less than about 0.001 mm to more than about 0.2 mm and they come in several different forms such as short fibers (known as staple, or chopped), continuous single fibers (filaments or monofilaments), untwisted bundles of continuous filaments (tow), and twisted bundles of continuous filaments (yam). Nonwoven webs can be formed by many processes such as meltblowing, spunbonding, solvent spinning, electrospinning, carding and airlaying. The basis weight of nonwoven webs is usually expressed in grams per square meter (g/m² or gsm).

“Comprise,” “comprising,” and “comprises”, as used herein, are open ended terms, each specifies the presence of what follows, e.g., a component, but does not preclude the presence of other features, e.g., elements, steps, components known in the art, or disclosed herein.

“Consisting essentially of”, as used herein, limits the scope of subject matter, such as that in a claim, to the specified materials or steps and those that do not materially affect the basic and novel characteristics of the subject matter. The term “Consisting of” further limits the scope to the specified elements, steps, or components.

“Substantially”, as used herein, means generally the same or uniform but allowing for or having minor fluctuations from a defined property, definition, etc. For example, small measurable or immeasurable fluctuations in a measured property described herein, such as viscosity, melting point, etc. may result from human error or methodology precision. Other fluctuations are caused by inherent variations in the manufacturing process, thermal history of a formulation, and the like. The compositions of the present invention, nonetheless, would be said to be substantially having the property as reported.

### Low Molecular Weight Metallocene-Catalyzed Polyolefin

The hotmelt adhesive of the invention comprises a low molecular weight metallocene-catalyzed polyolefin having a peak molecular weight below 130,000 g/mol. The peak molecular weight may be for example in the range of from 5,000 g/mol to 130,000 g/mol. The peak molecular weight is measured as indicated further below in the experimental section according to the Peak Molecular Weight (Mp) Measurement Method.

As for any of the components indicated in the claims, the hotmelt composition may comprise one, or a blend of two or more, of such low molecular weight metallocene-catalyzed polyolefins. Thus, unless indicated otherwise, when using the term “a low molecular weight metallocene-catalyzed polyolefin” it is meant the “at least one low molecular weight metallocene-catalyzed polyolefin(s)”.

The hotmelt adhesive typically comprises from 10% to 70% by weight of the low molecular weight metallocene-catalyzed polyolefin (or mixture thereof), in particular from 30% to 60% by weight of the low molecular weight metallocene-catalyzed polymer(s).

Metallocene-catalyzed polyolefins typically have a regular spatial repeat monomer unit distribution and a narrow molecular weight distribution, as is known in the art. Propylene-based metallocene-catalyzed polyolefins may be in particular used. The propylene-based metallocene-catalyzed polyolefins may be homopolymers or copolymers, in particular propylene-ethylene copolymers. Metallocene-catalyzed polyolefins useful in the present invention can be described as low- or semi-crystalline with a Heat of Crystallization, as measured according to the Heat of Crystallization Test Method described below, typically ranging of from 5 J/g to 45 J/g.

The propylene-ethylene copolymers comprise at least 50% by weight of the copolymer of propylene unit, in particular at least 60%, or at least 70%, or at least 80% by weight. The remaining monomers are ethylene monomers, and optionally other alpha olefin monomers may be present in the co-polymers, for example 4-methyl-1-pentene, pentene-1, 2-methylpentene-1, 3-methylbutene-1, heptene-1, dimethylpentene-1, trimethylbutene-1, ethylpentene-1, methylpentene-1, trimethylpentene-1, methylethylpentene-1, 1-octene, diethylbutene-1, propylpentane-1, decene-1, methylnonene-1, nonene-1, trimethylheptene-1, methylethylbutene-1, dodecene-1, and hexadodecene-1, and combinations thereof. The exact monomer distribution is typically published by the supplier, but can also be determined by a suitable method, such as nuclear magnetic resonance or infrared spectroscopies.

Suitable metallocene-catalyzed propylene-ethylene copolymers are commercially available from Clariant under the polymer range Licocene® PP, with a broad range of properties such as molecular weight, viscosity, crystallinity, etc. US2016/053,149A1 assigned to Clariant also describes suitable copolymers and on page 5 indicates that these examples were produced by the processes indicated in EP571,882. For a given catalyst system and given comonomer ratio, the molecular weight was regulated via the hydrogen partial pressure as molar mass regulator.

The low molecular weight metallocene-catalyzed polyolefin may comprise a blend of two copolymers, in particular:
a first low molecular weight metallocene-catalyzed propylene-ethylene copolymer having a Heat of Crystallization below 20 J/g; and
a second low molecular weight metallocene-catalyzed propylene-ethylene copolymer having a Heat of Crystallization above 20 J/g.

While not wishing to be bound by theory, it is believed that the crystallinity of the low molecular weight metallocene based polymer(s), which is/are the backbone of the formula, can be considered when formulating a composition according to the invention.

The first low molecular weight metallocene-catalyzed propylene-ethylene copolymer may have a Heat of Crystallization of less than 20 J/g, in particular from 5 J/g to 15 J/g, and may described as low-crystalline. The Heat of Crystallization is measured according to the Heat of Crystallization Test Method described below. A commercial example of the first copolymer is Licocene® PP 1602 from Clariant. Licocene PP 1602 is sold as granules and is described as a low melting, metallocene-technology based propylene-ethylene copolymer, which exhibits a low degree of crystallinity. The Mp of Licocene® PP 1602 was measured to be 75,900 g/mol and its Heat of Crystallization of 16.7 J/g (see measurement method below). Another example is Licocene® PP 1302. The Mp of Licocene® PP 1302 was measured to be 24,100 g/mol and its Heat of Crystallization of 11.8 J/g. Preferably, the first low molecular weight metallocene-catalyzed propylene-ethylene copolymer has a viscosity at 170° C. below 500 mPa.s. This particularly enables to avoid the use of mineral oil or any other plasticizer while still keeping the viscosity sufficiently low for slot applications. Licocene® PP 1302 has a viscosity of 173 mPa.s at 170° C. and a viscosity of 108 mPa.s at 190° C.

The second low molecular weight metallocene-catalyzed propylene-ethylene copolymer has a higher Heat of Crystallization than the first copolymer, of at least 20 J/g, in particular from 25 J/g to 45 J/g. Polymer in this range can be described as semi-crystalline. The second copolymer may have a Mp in the range of from 50,000 g/mol to 130,000 g/mol, or from 60,000 g/mol to 110,000 g/mol. A commercial example of the second copolymer is Licocene® PP 3602 which is sold as granules and is described as a low crystalline metallocene-catalyzed propylene-ethylene copolymer. Licocene® 3602 has a measured Heat of Crystallization of 35.0 J/g.

The first and second copolymers described above may be typically blended at a weight ratio of 10:90 to 90:10, for example 50:50 or 2:1 or 1:2. Blending two lower molecular weight copolymers with different crystallinity was found to enable high toughness (as generally required for bonds between fibrous materials like nonwovens or cellulose fibers) while at the same time keeping the viscosity in an acceptably low range. The above described ratios were found to enable the desired balance of toughness (controlled by the second copolymer) and viscosity (controlled by the first copolymer).

An example is a blend of Licocene® PP 3602 and Licocene® PP 1302, which are both propylene-ethylene copolymers from Clariant. Licocene 3602 is a relatively highly crystalline polymer while Licocene® PP 1302 has a medium crystallinity. In a blend of both (e.g. 2:1 for the ratio of Licocene® PP 1302 to Licocene® PP 3602) the overall crystallinity can be adjusted in a way that the resulting hotmelt adhesive has a low enough stiffness as required for strong NW-Film bonds but still a high Toughness (see experimental section below for a more detailed discussion of the Toughness).

While blending low molecular weight metallocene-catalyzed polyolefins may be useful, this is however not required in the present invention. However, in this respect, a “building block” of Licocene® PP 1302 and Licocene® PP 3602 in a 2:1 ratio is believed to be superior to using pure Licocene® PP 2502. The 2:1 blend of 1302 and 3602 has a lower crystallinity and hence lower stiffness than Licocene® PP 2502, while the higher peak molecular weights of 1302 and 3602 (75,900 and 80,000 g/mol), compared to 2502 (57,100 g/mol), compensate on the Toughness.

The low molecular weight metallocene-catalyzed polyolefin may also consist of a single low molecular weight metallocene-catalyzed polyolefin, in particular a propylene-ethylene copolymer. While Licocene® PP 2502 with a Heat of Crystallization of 29.4 J/g and a peak molecular weight of 57,100 g/mol can be used for that purpose, it is less preferred than the above described blend of two copolymers Instead, particularly suitable low molecular weight metallocene-catalyzed polyolefins (especially propylene-ethylene copolymers) for use as a single low molecular weight metallocene-catalyzed polyolefin have a Heat of Crystallization in the range of 20 J/g to 30 J/g and a peak molecular weight between 25,000 and 35,000 g/mol. An example of such low molecular weight metallocene-catalyzed polyolefin is Licocene® PP 2402 from Clariant. This propylene-ethylene copolymer is compatible with high molecular weight polyolefins while keeping the viscosity low enough for practical application, in particular in a formulation that is substantially free of a mineral oil. Licocene® PP 2402 is a low molecular weight metallocene-catalyzed polyolefin, which has a Heat of Crystallization of about 24 J/g, a peak molecular weight (Mp) of about 28,000 g/mol and a viscosity at 150° C. of about 2,000 mPa.s.

Vistamaxx grades, like Vistamaxx 8880 and Vistamaxx 8780 from Exxon are available as low molecular weight metallocene-catalyzed polyolefin, but Licocene grades, as described above which have a higher Toughness are preferably used in the present invention.

### High Molecular Weight Polyolefin

According to the invention, the inventors found that, when a polyolefin having a high peak molecular weight Mp of from 130,000 g/mol to 700,000 g/mol is used, then the Toughness of the formulation can be significantly increased. The high molecular weight polyolefin may have a peak molecular weight which is at least greater by 10,000 g/mol than the peak molecular weight of the low molecular weight metallocene-catalyzed polyolefin(s) described above (taking the highest peak if blends are used for the low Mp polymers), in particular at least 20,000 g/mol greater, or even at least 50,000 g/mol greater. The high molecular weight polyolefin may in particular have a peak molecular weight of from 140,000 g/mol to 410,000 g/mol, or from 150,000 g/mol to 360,000 g/mol.

The inventors have found that, surprisingly, the addition of a longer molecular weight polyolefin significantly increases the strain hardening of the blend besides increasing the elongation at break, which in combination results in a significantly higher Toughness of the formulation. Strain hardening is believed to be a “self-repairing mechanism of the blend when being strained, which avoids early rupture.

The high molecular weight polyolefin may advantageously consist of a single material to simplify the compounding and formulation of the hotmelt adhesive, but it is not excluded that it may also be a blend of individual materials falling under this definition. As for any of the components indicated in the claims, the hotmelt composition may comprise one, or a blend of two or more, of such high molecular weight polyolefin(s). Thus, unless indicated otherwise, when using the term “a high molecular weight polyolefin” it is meant the “at least one low molecular weight polyolefin(s)”.

The hotmelt adhesive may typically comprise from 1% to 20% of such a high molecular weight polyolefin (or mixture thereof), by weight of the hotmelt adhesive, in particular from 2% to 15%, especially from 5% to 15% by weight of the hotmelt adhesive. It is believed that already small additions of the higher molecular weight polyolefins can significantly boost the strain hardening and hence the Toughness. More than 10% may on the other hand increase the viscosity. Toughness, strain hardening and Elongation at break are measured and observed in the Extensional Test Method, submitting the adhesive to large deformations, as relevant when the bond is subjected to forces in use.

The high molecular weight polyolefin(s) is preferably a propylene copolymer. The copolymer may comprise different alpha olefin monomers such as ethylene, propylene, 4-methyl-1-pentene, pentene-1, 2-methylpentene-1, 3 -methylbutene-1, heptene-1, dimethylpentene-1, trimethylbutene-1, ethylpentene-1, methylpentene-1, trimethylpentene-1, methylethylpentene-1, 1-octene, diethylbutene-1, propylpentane-1, decene-1, methylnonene-1, nonene-1, trimethylheptene-1, methylethylbutene-1, dodecene-1, and hexadodecene-1, and combinations thereof.

Nonlimiting examples of commercially available high molecular weight polyolefins are Affinity EG 8200G, Engage 8200, Infuse 9817, Vistamaxx 3000, Vistamaxx 6102, Vistamaxx 6202, Vistamaxx 6502, VERsify 4200, VERsify 4301.

The high molecular weight polyolefin may in particular comprise or consists of a propylene-ethylene copolymer. The high molecular weight polyolefin may also be a metallocene-catalyzed based copolymer, in particular a metallocene-catalyzed propylene-ethylene copolymer. The high molecular weight polyolefin may in particular be a propylene-ethylene copolymer comprising greater than 80 wt. % of polypropylene units with isotactic stereochemistry. Examples of such copolymers are commercially available as the Vistamaxx series from ExxonMobil. For example, Vistamaxx 6202 and Vistamaxx 6502 are sold as pellets and are described by their manufacturer as primarily composed of isotactic propylene repeat units with random ethylene distribution, produced using a metallocene catalyst technology. Vistamaxx 6202 and 6502 were used as high molecular weight polymer in the formula examples below. Vistamaxx 6502 has the lowest viscosity, and thus the least impact on increasing the viscosity of the total composition.

### Tackifier

The hotmelt adhesive comprises a tackifier (or a mixture of tackifiers). The hotmelt adhesive typically comprise from 10% to 60%, in particular from 15% to 65%, or from 15% to 60%, or 30% to 60%, or 35% to 60% by weight of the composition, of the tackifier(s). Tackifiers, otherwise called “tackifier resins” or “tackifying resins”, are low-molecular weight compounds (oligomers) that are added to adhesive formulations to improve tack and peel adhesion materials. Usual tackifiers known in the art may be used in the present invention. Typical tackifiers are thermoplastic materials stable at least up to 200° C., being amorphous glasses at room temperature, and having a Tg higher than 50° C., preferably comprised between 80° C. and 125° C. Tackifiers typically have a molecular weight comprised between 500 and 2000 Daltons.

Tackifiers are in general organic chemicals with polycyclic structure. Commonly used tackifiers are selected from rosin resins and their derivatives (rosin esters), hydrocarbon resins produced from petroleum-based by-products of naphtha crackers, and terpene resins (modified or not). Hydrocarbon resins may be aliphatic, cycloaliphatic and aromatic resins (in particular C5 aliphatic resins, C9 aromatic resins, and C5/C9 aliphatic/aromatic resins), and may be optionally hydrogenated hydrocarbon resins.

Exemplary tackifiers include aliphatic hydrocarbon resins, aromatic modified aliphatic hydrocarbon resins, hydrogenated poly-cyclopentadiene resins, poly-cyclopentadiene resins, gum rosins, gum rosin esters, wood rosins, wood rosin esters, tall oil rosins, tall oil rosin esters, poly-terpenes, aromatic modified poly-terpenes, terpene-phenolics, aromatic modified hydrogenated poly-cyclopentadiene resins, hydrogenated aliphatic resins, hydrogenated aliphatic aromatic resins, hydrogenated terpenes and modified terpenes, and hydrogenated rosin esters. Particularly suitable tackifiers are rosin (and its derivatives) resins and hydrogenated hydrocarbon tackifiers, which are solid at room temperature. The tackifier(s) in the hotmelt composition may preferably comprise or consist of hydrogenated hydrocarbon tackifier(s).

The tackifier is preferably at least partially hydrogenated, preferably fully hydrogenated. Without wishing to be bound by theory, the inventors believe that a partially or particularly fully hydrogenated tackifier enables better compatibility with the other components of the adhesive composition of the present invention. Also, a fully hydrogenated tackifier is preferred due its lower tendency to deteriorate the odor of the adhesive formulation and hence the absorbent article.

The inventors found that the combination of one or more high molecular weight polyolefins and the additional presence of the tackifier enable higher Toughness values for the hotmelt composition.

### Compounding and Optional Ingredients

The hotmelt adhesive can be prepared by heating the polyolefins at a sufficiently elevated temperatures (e.g., about 135° C. to about 175° C.) to melt the copolymers. The tackifier and other ingredients (e.g., additive or other polymers) can be added to this molten primary polymer blend. A mixer can be used to mix the polymers and other additives together into a final hotmelt adhesive.

The resulting blend is cooled and conditioned for transport and storage. During application, the hotmelt adhesive is molten again and can be applied to a substrate using any known applicator devices, in particular slot coating which is a contact applicator.

The hotmelt adhesive according to the invention preferably has a viscosity at 170° C. is in the range from about 1,000 mPa·s to about 7,000 mPa·s, as measured according to the Viscosity Test Method as described herein.

There are significant advantages to minimizing or avoiding the use of a mineral oil. This can reduce the cost of the hotmelt adhesive, as well as eliminate an additional ingredient and potential issues that may be associated with supplying the additional ingredient.

The formulation of the present invention is advantageously substantially free of mineral oils and comparable plasticizers. The inventors have found that the absence of plasticizers enables both higher toughness and the advantageous long open times, which again enable an effective anchoring into the second substrate. Without not wishing to be bound by theory, the inventors believe that the absence of plasticizers slows down processes of crystallization of parts of the polyolefin components, which starts after application of the formulation onto the first substrate; crystallization in polyolefin blends is a diffusion driven process, which is accelerated via the presence of liquid low molecular weight components.

The hotmelt adhesive is also preferably free of other amorphous components, in particular polyolefins having a Heat of Crystallization of less than 5 J/g. While these can serve like a mineral oil to dilute the hotmelt composition in order to reduce the viscosity, these compounds also decrease at the same time the Toughness of the hotmelt of the present invention. Higher Toughness is advantageous especially for the nonwoven-nonwoven bonds in the core wrap bonds.

The hotmelt adhesive may optionally comprise an antioxidant. Non-limiting examples of suitable antioxidants include amine-based antioxidants such as alkyl diphenyl amines, phenyl-naphthylamine, alkyl or aralkyl substituted phenyl-naphthylamine, alkylated p-phenylene diamines, tetramethyl-diaminodiphenylamine and the like; and hindered phenol compounds such as 2,6-di-t-butyl-4-methylphenol; 1,3,5-trimethyl-2,4,6-tris(3′,5′-di-t-butyl-4′-hydroxybenzyl)benzene; tetra kis [methylene(3,5-di-t-butyl-4-hydroxyhydrocinnamate)]methane (e.g., IRGANOX™ 1010, from Ciba Geigy, New York); octadecyl-3,5-di-t-butyl-4-hydroxycinnamate (e.g., IRGANOX™ 1076, commercially available from Ciba Geigy) and combinations thereof. When used, the amount of the antioxidant in the hotmelt adhesive can be respectively less than 1%, alternatively from about 0.05% to about 0.75%, and alternatively from about 0.1% to about 0.5%, by weight of the hotmelt adhesive.

The hotmelt adhesive may optionally comprise a UV stabilizer that may prevent or reduce the degradation of the composition by radiation. Any UV stabilizer known to a person of ordinary skill in the art may be used in the hotmelt adhesive. Non-limiting examples of suitable UV stabilizers include benzophenones, benzotriazoles, aryl esters, oxanilides, acrylic esters, formamidine carbon black, hindered amines, nickel quenchers, hindered amines, phenolic antioxidants, metallic salts, zinc compounds, and combinations thereof. Where used, the amount of the UV stabilizer in the hotmelt adhesive can be less than 1%, alternatively from about 0.05% to about 0.75%, and alternatively from about 0.1% to about 0.5%, by weight of the hotmelt adhesive.

The hotmelt adhesive may optionally comprise a brightener, colorant, and/or pigment. Any colorant or pigment known to a person of ordinary skill in the art may be used in the hotmelt adhesive. Non-limiting examples of suitable brighteners, colorants, and/or pigments include fluorescent materials and pigments such as triazine-stilbene, coumarin, imidazole, diazole, titanium dioxide and carbon black, phthalocyanine pigments, and other organic pigments such as IRGAZINB, CROMOPHTALB, MONASTRALB, CINQUASIAB, IRGALITEB, ORASOLB, all of which are available from Ciba Specialty Chemicals, Tarrytown, N.Y. Where used, the amount of the brightener, colorant, and/or pigment in the hotmelt adhesive can be less than 10%, alternatively from about 0.01% to about 5%, and alternatively from about 0.1% to about 2%, by weight of the hotmelt adhesive.

The hotmelt adhesive may optionally comprise a fragrance such as a perfume or other odorant. Such fragrances may be retained by a liner or contained in release agents such as microcapsules that may, for example, release fragrance upon removal of a release liner from or compression on the adhesive composition. Where used, the amount of the fragrance in the hotmelt adhesive can be less than 3%, alternatively less than 2%, alternatively less than 1%, alternatively from about 0.05% to about 0.75%, and alternatively from about 0.1% to about 0.5%, by weight of the hotmelt adhesive.

The hotmelt adhesive may optionally comprise a wax or nucleation agent to accelerate the time until which it builds its full strength in the final bond, after the bond between the first and the second substrate has been generated (“setting time”). An example of such a wax, which is still compatible with the formulation and does not detract from the desired high toughness value, can be Licocene® PP 6102 from Clariant, which can be added by up to 5% of the total composition. The inventors found, however, that addition of such wax is not needed in the present invention because the setting time was found to be sufficiently short (at maximum 1 hour after forming of the bond), while the addition of a wax or nucleation agent also shortens the “open time” of the adhesive, i.e. the time span after application of the adhesive onto the first substrate during which it is still capable of effectively bonding to the second substrate, and the long open times of the formulations of the invention was found to contribute to the high performance in the described bonds. Therefore, hotmelt compositions of the present invention are preferably free of wax or nucleation agent.

### Renewable Materials

Any of the ingredients of the hotmelt composition may be at least partially obtained from renewable sources, in particular any of the component or the hotmelt composition as a whole may have a bio-based content of at least 50%. “Bio-based content” refers to the amount of carbon from a renewable resource in a material as a percent of the mass of the total organic carbon in the material, as determined by ASTM D6866-10, method B.

The metallocene catalyzed polyolefins used in the present invention can be used with significant (at least 50%) bio-based content. The Licocene grades from Clariant can be used in the renewable-based version under the trade-name Terra. So, instead of the grade “Licocene PP 1302” the grade “Licocene PP 1302 Terra” can be used.

### Examples & Data

Table 1 discloses the peak molecular weight (Mp) in g/mol of some commercially available polymers that may be used in the invention.

TABLE 1

| | Mp |
|---|---|
| Licocene PP 1302 | 24,100 |
| Licocene PP 1602 | 75,900 |
| Licocene PP 2402 | 28,470 |
| Licocene PP 2502 | 57,100 |
| Licocene PP 3602 | 80,000 ¹ |
| Vistamaxx 3000 | 299,500 |
| Vistamaxx 6102 | 687,700 ¹ |
| Vistamaxx 6202 | 214,104 |
| Vistamaxx 6502 | 185,300 |

¹ correlated (not measured directly)

Table 2 discloses the Heat of Crystallization in J/g of some commercially available polymers that may be used in the invention:

TABLE 2

| | Heat of Crystallization (J/g) |
|---|---|
| Licocene PP 1302 | 11.8 |
| Licocene PP 1602 | 16.7 |
| Licocene PP 2402 | 23.8 |
| Licocene PP 2502 | 29.4 |
| Licocene PP 3602 | 35.0 |

Table 3 shows exemplary formulations according to the invention (all ingredient values indicated in weight percent). Licocene® are propylene-ethylene copolymers from Clariant. Eastotac and Escorez are tackifiers available from Eastman and ExxonMobil respectively. Vistamaxx® is a polypropylene polymer from ExxonMobil primarily composed of isotactic propylene repeat units with random ethylene distribution.

TABLE 3

| | Licocene | Licocene | Licocene | Licocene | Licocene | Eastotac | Henghe | Henghe | |
|---|---|---|---|---|---|---|---|---|---|
| | PP | PP | PP | PP | PP | Resh H- | HM | H5 | Vistamaxx |
| Example | 2502 | 1602 | 1302 | 3602 | 2402 | 100L | 1000 | 1000 | 6502 |
| 1 | 50 | | | | | 40 | | | 10 |
| 2 | | 46.7 | | 23.3 | | 20 | | | 10 |
| 3 | | 33.3 | | 16.7 | | 40 | | | 10 |
| 4 | | 36.7 | | 18.3 | | 40 | | | 5 |
| 5 | | | 24 | 40 | | 26 | | | 10 |
| 6 | | | | | 50 | | 40 | | 10 |
| 7 | | | | | 50 | | | 40 | 10 |

The adhesives of the invention may preferably have a high Toughness value (at least 11 MJ/m³ at 37° C., and preferably at least 25 MJ/m³ at 37° C.) and Yield Stress (at least 0.7 MPa, and preferably at least 1.2 MPa at 37° C.) and optionally a high Storage Modulus G′ (at least 3 MPa at 37° C., and preferably at least 5.0 MPa at 37° C.). The method (extensional rheology) used to measure Toughness and Yield Stress enables to screen adhesives with regards to their resistance to large strains, which occur under the real load case in use, as opposed to standard rheological adhesive tests like oscillatory rheology (yielding e.g. the storage modulus G′), which only investigates the behavior of adhesives under small deformations. Toughness and Yield Stress therewith provide critical complementary information over the storage modulus, which only describes the elastic resistance to initial small deformations and is indicative of the “stiffness” of an adhesive.

The open time of each exemplary composition was assessed using the Cross Over Temperature [° C.] (see Oscillatory Rheometry Test Method), from hot to cold, which measures the temperature at which the hotmelt adhesive solidifies when cooling down. A lower Cross Over Temperature correlates with a longer open time for the hotmelt adhesive because, for a given application temperature (typically 160° C.), it takes a longer time for the adhesive at a given basis weight to cool down to reach the Cross Over Temperature. Also shown are the Toughness, the Storage modulus (G′) and the Yield Stress, with the latter two being measures of the stiffness of the hotmelt adhesive.

Comparative hotmelt examples were tested under the same conditions. The combined resulted in Table 4 below.

TABLE 4

| | | | Storage | Crossover |
|---|---|---|---|---|
| | Toughness | Yield Stress | Modulus (G′) | Temperature |
| Adhesive | [MJ/m³] | [MPa] | [MPa] | [° C.] |
| (Source) | @ 37° C., 1 s⁻¹ | @ 37° C., 1 s⁻¹ | @ 37° C. cold to hot | hot to cold |
| Requirement | ≥11 | ≥0.7 | ≥3.0 (optional) | <75 |
| Preferred | ≥25 | ≥1.2 | ≥5.0 (optional) | <70 |
| Requirement | | | | |
| Example 1 | 13.8 | 3.2 | 12.1 | 53 |
| Example 2 | 22.4 | 1.5 | 5.6 | 63 |
| Example 3 | 43.5 | 1.2 | 3.9 | 47 |
| Example 4 | 22.5 | 1.3 | 4.3 | 53 |
| Example 5 | 40.2 | 2.0 | 8.5 | 59 |
| Example 6 | 65.0 | 2.0 | 5.0 | 48 |
| Example 7 | 40.0 | 2.2 | 4.2 | 48 |
| NW 1414 | 23.5 | 1.0 | 8.4 | 94 |
| (HB Fuller) | | | | |
| (comparative) | | | | |
| DM 3800 | 22.0 | 0.5 | 2.7 | 98 |
| (Henkel) | | | | |
| (comparative) | | | | |
| Licocene 2502 | 9.0 | 6.6 | 33.5 | 73 |
| (Clariant) | | | | |
| (comparative) | | | | |

Example 5 has an application friendly viscosity of 6,200 mPa.s at 170° C.

Example 6 has a very high Toughness value, while also having a relatively low viscosity of 3,300 mPa.s at 170° C. and 6,200 mPa.s at 150° C. Example 7 has a comparably low viscosity of 3,200 mPa.s at 170° C. These examples show that Licocene PP 2402 may be used as the low molecular weight metallocene-catalyzed polyolefin, and provide a relatively high Toughness and long open time on one hand, while still having relatively low viscosity that enables a lower application temperature for the hotmelt adhesives. Thus, the hotmelt adhesive may optionally comprise a single low molecular weight metallocene-catalyzed polyolefin, in particular wherein the low molecular weight metallocene-catalyzed polyolefin has a Heat of Crystallization in the range of 20 J/g to 30 J/g and a peak molecular weight between 25,000 and 35,000 g/mol.

The invention also allows cost-saving by decreasing the amount of adhesive used vs. conventional adhesives and/or allows improved performance at same amount usage vs. conventional adhesives.

While not wishing to be bound by theory, the inventors believe the Toughness parameter is predictive of peel creep resistance in construction bonds and creep resistance in constant displacement tests as used for elastic attachment adhesives. The inventors also found that the Toughness parameter is indicative of the usage reduction potential of an adhesive. The higher the Toughness parameter, the less usage of the adhesive is possible for an adhesive, without compromises in creep resistance. While the inventors believe that there is no theoretical upper limit to the Toughness (e.g. up to 60 MJ/m³), there may be an upper limit to yield stress at 37° C. (around 20 MPa) and for G′ at 37° C. (around 50 MPa) as of which the adhesive may become too brittle.

The inventors found that the same principles, as underlying to strong nonwoven-nonwoven bond bonds, applies to bonds between nonwoven and cellulose fibers, as used in the patch. The underlying concept is the creation of a “mechanical lock”, by “cementing” the fibers of the nonwovens respectively the cellulose patch in the adhesive with high toughness. This requires, of course and excellent enwrapping of the individual fibers of both substrates with the adhesive. To ensure that the adhesive is able to sufficiently engage and anchor to the fibers of the secondary substrate in the process, it needs to have a sufficiently long open time, as the adhesives of the present invention.

The inventors have found that the long open time contributes, in addition to the high Toughness, to the very strong performance in the bonds of the present invention. The long open time enables an excellent anchoring of the adhesive into the secondary substrate at the combining point of primary and secondary substrate. The inventors believe that the strong bond performance is enabled by a “mechanical lock” effect: the combination of high wrap angles of the adhesive around the fibers to be bonded (preferably above 180°) with the high values of Toughness, Yield Stress and high Storage Modulus at 37° C. enable the observed strong bond performance. In the formulation, the long open time is enabled by the preference of primarily propylene based polymers (which need in general a longer time for nucleation and crystal growth due to steric hindrance than primarily ethylene based polymers) and the absence of plasticizers like mineral oils.

In the below examples, the adhesives were slot coated with 1 mm wide stripes/1 mm gap between the stripes at a specified basis weight (on the stripes) between two nonwovens. As only half of the area was covered, the average basis weight over the whole area is half of the basis weight on the stripes. A static Peel Hang Test was conducted. Rectangular test specimens were prepared with a width of 25.4 mm perpendicular to the adhesive stripes and a delamination length of 6 mm. The delamination length is the distance along which the weight travels down during the test until the specimens are delaminated. The specimens were hanged with one nonwoven maintained in vertical position and the other nonwoven attached to a weight of 150 g.

The delamination time (“Peel Hang Time”) was measured 10 times per test option (adhesive/basis weight combination) and the average reported. The Peel Hang Time is representative of the resistance of the adhesives to peel creep forces.

TABLE 5

| | Average Basis | Peel Hang Time at 22° C. in |
|---|---|---|
| Adhesive | weight [g/m²] | minutes (max 1000 minutes) |
| DM 3800 | 2 | 324 |
| DM 3800 | 1 | 65 |
| DM 3800 | 0.8 | 66 |
| NW 1414 | 2 | 888 |
| NW 1414 | 1 | 107 |
| NW 1414 | 0.8 | 78 |
| Ex 5 | 2 | 999 |
| Ex 5 | 1 | 938 |
| Ex 5 | 0.8 | 363 |
| Ex 7 | 0.8 | 909 |

The data show that the inventive adhesives have better performance against peel creep forces, even at lower basis weight than conventional prior art adhesives. The data also show the advantageous effect of high Toughness, and specifically the combination of high Toughness and long open time.

### General Description of an Absorbent Article 20

“Absorbent article”, as used herein, refers to personal hygiene products that are placed against or in proximity to the body of the wearer to absorb and contain the various exudates discharged from the body. Absorbent articles include baby diapers, training pants, adult incontinence undergarments, feminine hygiene products, and the like. As used herein, the term “body fluids” or “body exudates” includes, but is not limited to, urine, blood, vaginal discharges and fecal matter.

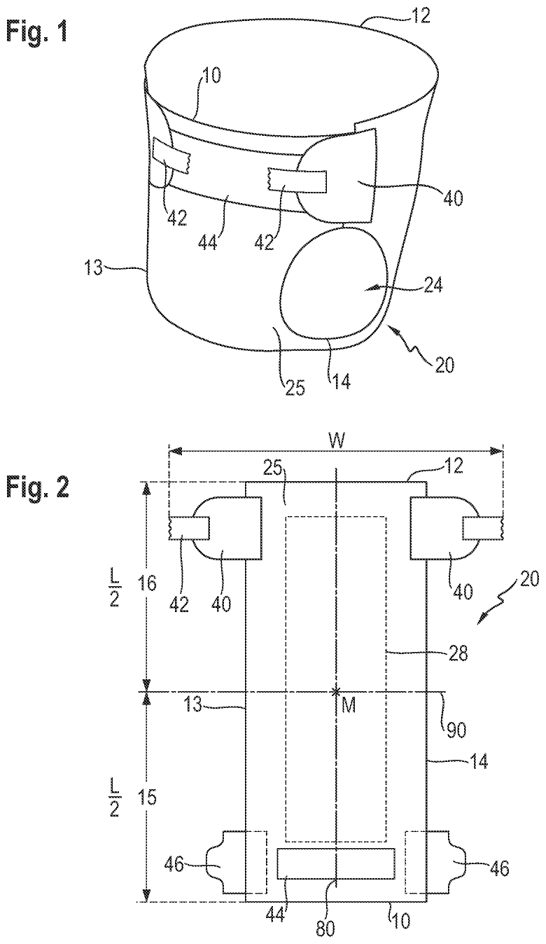

An exemplary absorbent article according to the invention in the form of a baby taped diaper 20 is represented in FIGS. 1-3. FIG. 1 is a perspective view of the exemplary diaper in a closed state as it would appear when worn by a wearer. This taped diaper 20 is shown for illustration purpose only, as the invention may be used for making a wide variety of diapers or other absorbent articles such as baby diaper pants, adult incontinence pants or feminine sanitary pads. In the following, the word “diaper” and “absorbent article” are used interchangeably. The Figures are used herein as illustration of one way to carry out the invention and are not limiting the scope of the claims, unless specifically indicated to do so.

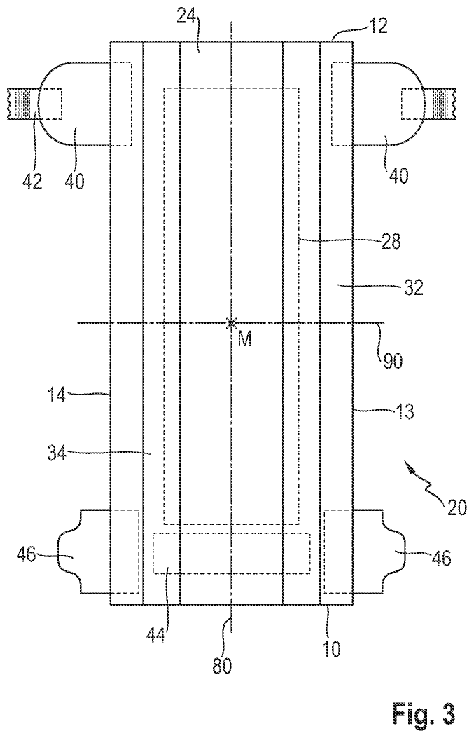

The absorbent article comprises a liquid permeable topsheet 24 on its wearer-facing surface, a liquid impermeable backsheet 25 on its garment-facing surface and an absorbent core 28 between the topsheet and the backsheet (shown in dotted line in FIGS. 2 and 3). The topsheet typically forms the majority of the wearer-contacting surface of the article and is the first layer that the body exudates contact. The topsheet is liquid permeable, permitting liquids to readily penetrate through its thickness. Any known topsheet may be used in the present invention. The backsheet typically comprises a fluid impermeable plastic film, which may be printed with a backsheet pattern, and a low basis weight nonwoven outer cover glued to this impermeable film to give a nicer feel and appearance to the backsheet.

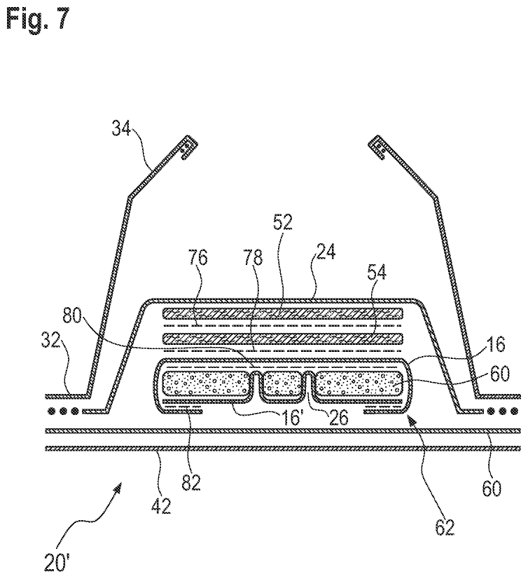

The absorbent article typically comprises a fluid acquisition layer 52 and optionally a distribution layer 54 between the topsheet 24 and the absorbent core 28, which are represented in the FIG. 7, as well as outer barrier cuffs 32 and inner barrier cuffs 34, as is known in the art. These cuffs typically comprise one or more elastic strands 36. Acquisition layers 52 are typically nonwovens, such as through-air bonded carded nonwovens, which may be hydrophillically treated.

The distribution layer 54, also known in the art as “patch”, comprises or consists of relatively loose fibers with no or weak intra-fiber bonds and is disposed between the acquisition layer 52 and absorbent core 28. When a nonwoven acquisition layer 52 is present in the article, the distribution layer 54 may be for example deposited on this acquisition layer, the two layers being further joined to absorbent core and the rest of the article, as is known in the art. Alternatively, the distribution layer 54 may also disposed directly between the topsheet 24 and absorbent core 28 if the article does not comprise an acquisition layer 52.

A typical example of such patch material comprises or consists of cross-linked cellulose fibers. The distribution layer may for example comprise at least 50% and up to 100% by weight of cross-linked cellulose fibers (including the cross-linking agents). The cross-linked cellulosic fibers may be crimped, twisted, or curled, or a combination thereof including crimped, twisted, and curled. The distribution layer comprising cross-linked cellulose fibers may comprise other fibers. The cross-linked cellulosic fibers provide higher resilience and therefore higher resistance against the compression in the product packaging or in use conditions, e.g. under baby weight. While the patch material may be comprised of cellulose fibers, in particular cross-linked cellulose fibers, other materials are of course possible, however the distribution layer is preferably free of superabsorbent polymer.

Examples of chemically cross-linked cellulosic fibers that have been used to make a distribution layer are disclosed in U.S. Pat. Nos. 5,549,791, 5,137,537, WO95/34329 or US2007/118087. This type of material has been used in the past in disposable diapers as part of an acquisition-distribution system, for example in US2008/0312622 A1 (Hundorf).

The absorbent article may also comprise other usual components if it is desired to increase the performance of the article, such as transverse barrier cuffs, front and/or back elastic waistbands, a lotion application on the topsheet, longitudinally extending channels in the core and/or the distribution layer, a wetness indicator, etc . . . all these components have been extensively described and exemplified in the art. More detailed disclosures of example of such components are for example disclosed in WO201493323, WO2015/183669 (both Bianchi et al), WO 2015/031225 (Roe et al.) or WO2016/133712 (Ehrnsperger et al.) to name a few.

The absorbent article typically comprises a front edge 10, a back edge 12, and two longitudinally-extending side (lateral) edges 13, 14. The front edge 10 is the edge of the article which is intended to be placed towards the front of the user when worn, and the back edge 12 is the opposite edge, and together form the waist opening of the diaper. The lateral edges 13, 14 respectively form the two leg openings. The topsheet 24, the backsheet 25, the absorbent core 28 and the other article components may be assembled in a variety of well-known configurations, in particular by gluing, fusion and/or pressure bonding. The absorbent articles of the invention may comprise any typical layers and components used in absorbent products of the diaper type, and which are not necessarily represented in the simplified FIGS. 1-3. A plurality of absorbent articles may be packaged together in a package.

Referring to FIGS. 1 and 2, the absorbent article 20 in the form of a taped diaper may have a discrete landing zone 44 on its garment-facing side, typically disposed proximate the front edge 10 of the article 20. The landing zone 44 is configured to receive the fasteners 42 and may comprise, for example, a plurality of loops configured to be engaged with, a plurality of hooks on the fasteners 46, or vice versa.

The landing zone 44 typically comprises one or more discrete nonwoven materials that are attached to a portion of the outer cover material 40 in the front waist region 12.

### General Description of an Absorbent Core 28

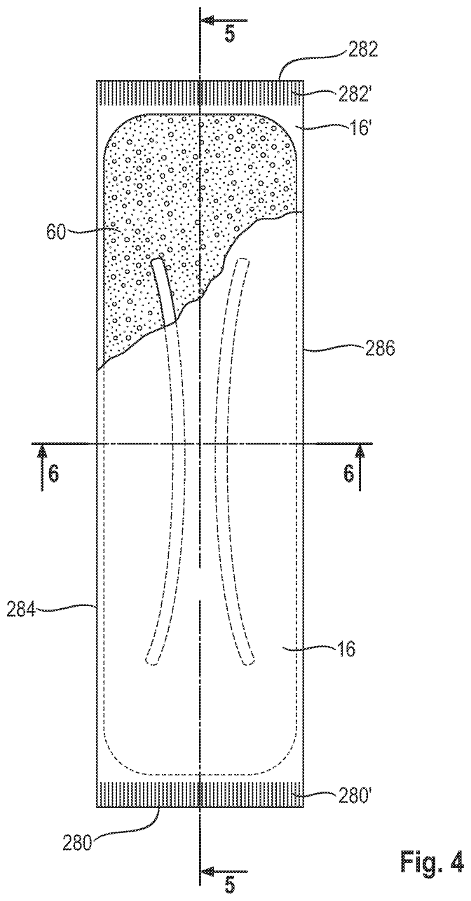

The absorbent core 28 is the component of the absorbent article having the most absorbent capacity. An exemplary absorbent core 28 is shown in isolation in FIGS. 4-6, in dry state (before use). The absorbent core may typically have a generally rectangular shape as defined by the longitudinal edges 284, 286 and transversal front edge 280 and back edge 282. The absorbent core 28 comprises an absorbent material 60, deposited as a layer having a generally rectangular outline, as represented on FIG. 4. This absorbent core represented is of course not limiting the scope of the invention as the invention is applicable to a wide variety of absorbent cores. It is also common to have an absorbent material 60 layer having a non-rectangular outline (“shaped” core), in particular the absorbent material layer may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “sand-hour” for the area of the absorbent material.

The absorbent material 60 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores where the absorbent material consists of SAP.

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from 20 g/g to 40 g/g. “Superabsorbent polymer particles”, as used herein, refers to a superabsorbent polymer material which is in particulate form so as to be flowable in the dry state.

Various absorbent core designs comprising high amount of SAP have been proposed in the past, see for example in U.S. Pat. No. 5,599,335 (Goldman), EP1,447,066 (Busam), WO95/11652 (Tanzer), US2008/0312622A1 (Hundorf), WO2012/052172 (Van Malderen). In particular, the SAP printing technology as disclosed in US2006/024433 (Blessing), US2008/0312617 and US2010/0051166A1 (both to Hundorf et al.) may be used. In these absorbent cores, two absorbent layers are combined to form the absorbent core 28. The invention is however not limited to a particular type of absorbent core. The absorbent core may also comprise one or more glue such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references.

The absorbent core 28 may also comprise an auxiliary adhesive 72 as shown in FIG. 7. The auxiliary adhesive 72 may be deposited on one or both of the first and second substrates 16 and 16′ before depositing the absorbent particulate polymer material 60 thereon, for enhancing adhesion of the superabsorbent particles. It may be preferable to deposit the auxiliary adhesive at least on the top core wrap layer, which is typically the most hydrophilic of the top and bottom layers, if the layers are made of differently hydrophillically treated material. The auxiliary glue 72 may also aid in immobilizing the absorbent particulate polymer material 60 and may comprise the same hotmelt adhesive of the invention as described hereinabove or may also comprise other or additional adhesives including but not limited to sprayable hotmelt adhesives.

The hotmelt adhesive may be applied in the absorbent particulate polymer material area at a basis weight of from about 2 g/m² to about 7 g/m² (gsm), in some embodiments from about 2 gsm to about 9 gsm, or from about 4 gsm to about 9 gsm. This may be a combined basis weight from application on a first and a second substrate, for example, 4 gsm and 3 gsm, respectively, or 5 gsm and 4 gsm, respectively. The front end seal may have from about 10 gsm to about 35 gsm of adhesive. Similarly, the back end seal may have from about 10 gsm to about 35 gsm of adhesive. In some embodiments, either or both of the front and back end seals may have from about 5 gsm to 15 gsm of adhesive.

As indicated previously, the absorbent material may be deposited as a continuous layer within the core wrap. The absorbent material may also be present discontinuously for example as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern wherein the resulting layer is substantially continuously distributed across the absorbent particulate polymer material area. As for example taught in US2008/312,622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

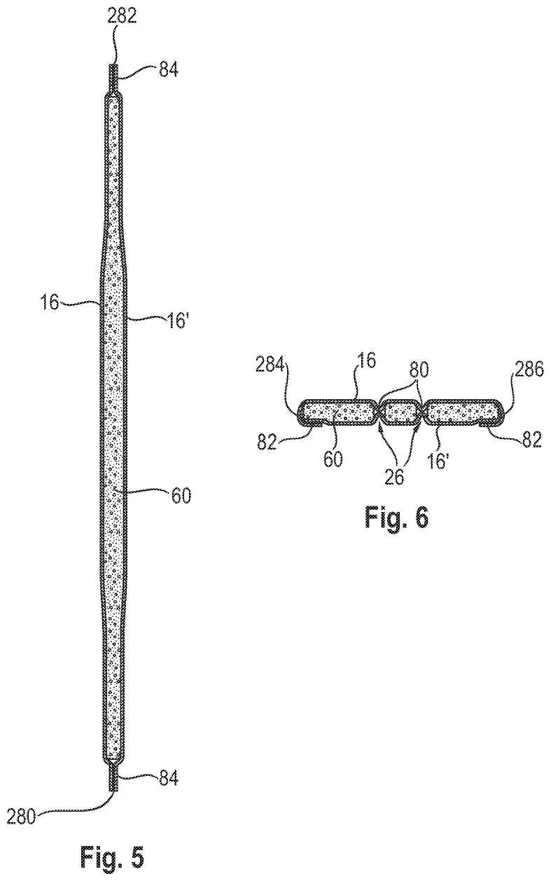

The basis weight (amount deposited per unit of surface) of the absorbent material may also be varied to create a profiled distribution of absorbent material, in particular in the longitudinal direction (as schematically illustrated in FIG. 5) to provide more absorbency towards the center and the middle of the core, but also in the transversal direction, or both directions of the core. The absorbent core may also comprise longitudinally extending channels which are substantially free of absorbent material within the absorbent material area. The core wrap may be bonded through these material-free areas. Exemplary disclosures of such channels in an airfelt-free core can be found in WO2012/170778 (Rosati et al.) and US2012/0312491 (Jackels). Channels may of course also be formed in absorbent cores comprising cellulose fibers.

### Core Wrap 16, 16′

The function of the core wrap is to contain the absorbent material. Different core wrap constructions can be used. A typical core wrap construction comprises two nonwoven substrates 16, 16′, which are attached to another and form respectively the top layer 16 and the bottom layer of the core wrap 16′. These two layers may be typically attached to another along at least part of the periphery of the absorbent core to form a seal. Typically, neither the first nor the second substrate needs to be shaped, so that they can be rectangularly cut for ease of production, but other shapes are not excluded. The terms “seal” is to be understood in a broad sense. The seal does not need to be continuous along the whole periphery of the core wrap but may be discontinuous along part or the whole of it, such as formed by a series of seal points spaced on a line. Typically, a seal may be formed by gluing and/or thermal bonding.

The core wrap represented in the Figures comprises a top layer 16 which is wider than the bottom layer 16′, so that two flaps extending from the top layer can be folded over the bottom layer along the longitudinal edges 284, 286 of the core respectively. The top layer and bottom layer are longitudinally bonded, typically by an adhesive, to form the longitudinal seals 82. The absorbent core's front edge 280 and back edge 282 may also be sealed, for example by a sandwich seal 84. Such transversal seals may for example made by adhesive stripes applied in machine direction by the slot glue technique, as is known in the art. Alternatively, is it possible to leave the transversal edges 280, 282 open without a seal. For example, there may be enough core wrap material between the edges of the core and the absorbent material 60 to provide a buffer zone at these ends.

The hotmelt adhesive of the invention is particularly applicable to form the longitudinal core wrap seals 82 as well as the end core bag seals 84, if present, as well as the core channel bonds 86 that will be discussed further below. Alternatively, the core wrap may be made of a single piece of nonwoven which has been folded over itself around the absorbent material layer 60, and is bonded to itself along a single longitudinal seal, instead of two longitudinal seals 82 as represented in the Figures. The invention is also applicable to such a single longitudinal core wrap seal.

The top layer 16 and the bottom layer 16′ may be made from the same base substrate material, but with a different treatment to modify its hydrophilicity. Such nonwoven substrate may have a basis weight within a range of from about 8 to about 12 gsm. The top layer may be typically a nonwoven layer made of synthetic fibers that has been treated with a surfactant to increase its hydrophilicity. The bottom layer may be made of synthetic fibers which are inherently hydrophobic. The top and bottom layers may each comprises or consists of a nonwoven web, such as a carded nonwoven, a spunbond nonwoven (“S”) or a meltblown nonwoven (“M”), and a multi-layer of any of these. For example, spunbond/meltblown laminate (spunmelt) polypropylene nonwovens are commonly used and are particularly suitable, especially those having a multi-layer SMS, or SMMS, or SSMMS, structure. Examples are disclosed in U.S. Pat. No. 7,744,576, US2011/0268932A1, US2011/0319848A1 or US2011/0250413A1. Typical material used to make the synthetic fibers are PE (polyethylene), PET (polyethylene terephthalate) and in particular PP (polypropylene).

Spunbond, also called spunlaid, nonwovens are made in one continuous process. Fibers are spun through a number of small orifices in a spinneret to form fibers or filaments, which are then directly dispersed into a web by deflectors or can be directed with air streams on a moving foraminous surface, such as a wire mesh conveyor. Meltblown nonwovens are produced by extruding melted polymer fibers through a spinneret or die consisting of up to 40 holes per inch to form long thin fibers which are stretched and cooled by passing hot air over the fibers as they fall from the die. The diameters of the fiber are significantly reduced by hot air which also breaks the continuous filaments into microfibers of varying length to diameter ratio. The extremely fine fibers (typically polypropylene) differ from other extrusions, particularly spunbond, in that they have low intrinsic strength but much smaller size offering key properties.

The spunbond process can be combined with the meltblown process to form a multi-layer web having S (spunbond) layer and M (meltblown) layer, in particular SM, SMS or SMMS webs, which are strong and offer the intrinsic benefits of fine fibers. The nonwovens may be consolidated using known techniques, typically thermal point bonding. In thermal point bonding, heat is applied locally on individual regions of the nonwoven to locally melt and fuse the fibers together. Fusion bond patterns are for example disclosed in US 2011/0250413 (Hu et al.) and US2014/0072,767A1 (Klaska et al.). The resultant web is typically collected into rolls at the supplier and subsequently converted to finished products.

### Core Channels

The absorbent core 28 may comprise one or more channels 26, in particular at least one channel on each side of the core's longitudinal centerline, which may or may not be connected and are present within the absorbent material layer. The channels may in particular be areas substantially free of absorbent material, in particular areas completely free of absorbent material (accidental minute amount of absorbent material due to involuntary contamination of the channels due to the high speed of the making process being disregarded).

The channels 26 may comprise a channel bond 86 between the top side 16 of the core wrap and the bottom side 16′ of the core wrap. This bond 86 provides for structural integrity of the channels in dry and wet state. Any known bonding techniques known in the art may be used to provide for this bond, but in particular a hotmelt adhesive bond may be used for the channel bond(s) 86. An adhesive may be for example applied in the areas of the channels on the inner side of the top side and/or the inner side of the bottom side of the core wrap, typically by slot glue application or any other means, followed by the application of pressure in the areas of the channels to provide a good adhesive bonding in these areas. Exemplary patent disclosures of such adhesive bonding processes can be found for an airfelt or airfelt-free absorbent cores in WO2012/170,798A1 (Jackels et al.), EP2,905,000 (Jackels et al.) and EP2,905,001 (Armstrong-Ostle et al.). The layer of adhesive forming the channel bonds may typically extend beyond the channel areas to form an auxiliary adhesive layer to help immobilizing the absorbent material on one of the internal side of the core wrap.

The hotmelt adhesive of the invention may be used to make these channel bonds 86, in addition or alternatively to the core perimeter bonds 82, 84. Typically, the channel bonds 86 may generally have the same outline and shape as the channel areas 26 in which they are contained, but may be slightly smaller to allow for a safety margin (e.g. by a few mm) as some deviations from the optimal registration may happen during high speed process. It is expected that the channel bonds 86 may be more efficiently made and stronger if they are provided in macroscopic areas with no absorbent material (except of course accidental contamination) compared to bonds provided in areas containing non-negligible absorbent material.

### Backsheet

The backsheet 25 is the liquid impermeable layer that generally form the garment-facing side of the absorbent article. The backsheet 25 prevents, or at least inhibits, the bodily exudates absorbed and contained in the absorbent core 28 from soiling articles such as bedsheets, undergarments, and/or clothing. The backsheet typically comprises a liquid impermeable, or at least substantially liquid impermeable layer, typically a plastic film, e.g. having a thickness of about 0.01 mm to about 0.05 mm. Suitable backsheet materials also include breathable materials which permit vapors to escape from the absorbent article, while still preventing, or at least inhibiting, bodily exudates from passing through the backsheet.

The backsheet 25 is typically a laminate comprising a plastic film and on its external side a nonwoven outer cover for improving the overall feel of the backsheet. The outer cover nonwoven (sometimes referred to as a backsheet nonwoven) is joined to and covers the backsheet film. Thus, the outer cover material typically forms at least a portion of the garment-facing surface of the absorbent article 20. The outer cover material may comprise a bond pattern, apertures, and/or three-dimensional features.

### Pant diaper

The absorbent article may also be in the form of a pant having permanent or refastenable side seams, which is not represented herein but for which the invention may also apply. Pant articles comprising refastenable seams are for example disclosed in US2014/0,005,020 and U.S. Pat. No. 9,421,137. Typical pant articles comprise a chassis (sometimes referred to as a central chassis or central panel) comprising a topsheet, a backsheet, and an absorbent core, which may be as disclosed herein, and a front belt that defines a front waist region, and a back belt that defines a back waist region. The chassis may be joined to a wearer-facing surface of the front and back belts or to a garment-facing surface of the belts. Side edges of the front belt may be joined to side edges of the back belt to form two side seams. The side seams may be any suitable seams known to those of skill in the art, such as butt seams or overlap seams, for example. When the side seams are permanently formed or refastenably closed, the absorbent article in the form of a pant has two leg openings and a waist opening circumference. The side seams may be permanently joined using adhesives or bonds, for example, or may be refastenably closed using hook and loop fasteners, for example.

Alternatively, instead of attaching belts to the chassis to form a pant, discrete side panels may be attached to side edges of the chassis. Suitable forms of pants comprising discrete side panels are e.g., disclosed e.g., in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Hotmelt Adhesive Application

The hotmelt compositions of the invention are particularly useful to form a core wrap bond or a patch-to-core bond. The hotmelt adhesive is typically applied in molten state on a first side of the core wrap. The patch or the second side of the core wrap is then contacted with the hotmelt adhesive, preferably with at least some pressure being applied between the two layers before the hotmelt composition solidifies to ensure that bonding takes place.

The hotmelt adhesive may be applied by any known process, which may be contact (slot, bead, adhesive coating as disclosed in WO2014/085,063A1) or non-contact (spraying with spiral or random pattern, including intermittent spray application). The hotmelt adhesive may be applied by any commercial applicators such as Nordson's Summit® (spiral), Signature® or Rhythm® applicator system. The hotmelt adhesive may be applied in a contact process (e.g., slot coating) or non-contact process on the first or the second substrate or both substrates preferably at a line speed of more than 2 m/s, in particular of more than 3 m/s, or even of more than 4 m/s.

The hotmelt adhesive may be typically applied at a basis weight ranging from about 0.5 gsm to about 30 gsm, alternatively from about 1 gsm to about 20 gsm, between the two layers in the areas to be bonded. The hotmelt adhesive may hold the first layer and the second layer bonded together within the bond area on its own. Alternatively, the hotmelt composition may be supplemented by another bonding means, such as mechanical bonds or fusion bonds.

### Test Methods

### Peak Molecular Weight (Mp) Measurement Method

The peak molecular weight is determined using a gel permeation chromatography (GPC) method. GPC is a well-known method wherein polymers are separated according to molecular size, the largest molecule eluting first. The peak molecular weights referred to herein can be determined with gel permeation chromatography (GPC) using polystyrene calibration standards, such as is done according to ASTM D5296. The molecular weight of any polymer or unknown polymer measured using GPC so calibrated is the styrene equivalent molecular weight, which herein is defined as the “peak molecular weight.” Suitable solvents and temperatures are employed with GPC in order to achieve adequate molecular weight separation and resolution.

### Heat of Crystallization Test Method

The Heat of Crystallization Parameter of a hotmelt adhesive is determined using the Heat of Crystallization Test Method, which consists of performing ASTM D3418-15 with the following additional guidance. Specimen(s) are preferably extracted from molded or pelleted raw material adhesive composition. If raw material is not available, specimen(s) of adhesive are extracted from bonds of interest in an absorbent article using techniques known to those of skill in the art. Dry nitrogen is used as the purge gas in the differential scanning calorimeter (DSC). The rate of increase of temperature in the DSC is 10° C./min, and the rate of decrease of temperature in the DSC is 1° C./min. The mass-normalized heat of crystallization is calculated as specified in section 11.4 based on the curve corresponding to decreasing temperature (at 1° C./min) and is reported as the “Heat of Crystallization” in units of joules per gram (J/g) to the nearest 0.1 J/g.

### Viscosity Test Method

The Viscosity Test method consists of performing a shear flow ramp on a rotational rheometer (such as ARES-G2, TA Instruments, New Castle, Del., USA, or equivalent). The rheometer is operated in a cone and plate configuration with a stainless steel cone 40 mm in diameter and with 0.04 rad cone angle mounted as upper tooling and stainless steel plate 40 mm in diameter as bottom tooling. Further the rheometer needs to be capable of sample temperature control with a precision equal to or better than 0.5° C. over at least the range of 20° C. up to 200° C.

A measurement gap of 49 μm is used in the method. To compensate for thermal expansion of the tooling the actual gap is mapped. For any temperature setpoint of interest, the following procedure is used (typical temperature setpoints of interest in this method include, but are not limited to 150° C., 170° C., and 190° C.) The rheometer is heated to the required measurement temperature. After 10 minutes of equilibration time the actual gap is determined by a “zero gap” routine. Zeroing the sample gap requires lowering the upper tooling until it touches the lower tooling, and an axial force is detected by the rheometer which is at least greater than 2 N. At this point the gap value is set to zero.

For a viscosity measurement at any temperature setpoint of interest, the compensation for thermal expansion is first determined as described above. The polymer composition is introduced in the rheometer, the gap is set to 74 μm, excess protruding sample is trimmed, and the gap is then set to 49 μm. The sample is preheated for 2 minutes at the temperature setpoint of interest. The shear stress is then recorded at 11 different shear rates logarithmically spanning the decade ranging from 1 and 10 s⁻¹, namely at shear rates of 1.00, 1.26, 1.58, 2.00, 2.51, 3.16, 3.98, 5.01, 6.31, 7.94, and 10.00 s⁻¹.

### Analysis

The data are plotted on log-log fashion with shear rate on the abscissa and shear stress on the ordinate (logarithmic scales). A linear fit is then performed. Starting at the high-shear-rate end of the range, at least six and as many consecutive points as possible are included such that an R2 value of 0.9 or greater results. If an R2 value of 0.9 cannot be achieved fitting only six points, the fit of the six points corresponding to the highest shear rates is accepted. The value of the slope is defined as the viscosity parameter, which is reported in millipascal seconds (mPa s) to the nearest hundred mPa s.

### Oscillatory Rheometry Test Method

The Oscillatory Rheometry Test Method is used to measure the Storage Modulus G′ and the Loss Modulus G″ of a polymer composition. A controlled-strain rotational rheometer (such as Discovery HR-3, TA Instruments, New Castle, Del., USA, or equivalent) capable of sample temperature control (using a Peltier cooler and resistance heater combination) with a precision equal to or exceeding 0.5° C. over at least the range of −10° C. to 150° C. The rheometer is operated in a parallel plate configuration with 20-mm stainless steel parallel-plate tooling.

A parallel plate gap of 1000 μm is initially used in the method. To compensate for thermal expansion of the tooling, the gap is set to 1000 μm, and a mapping of actual plate gap (as measured using a suitable standard test fluid) a function of temperature over the range −10° C. to 150° C. is performed. This mapping is then used throughout the determination of the Storage Modulus Parameter and the Loss Modulus Parameter.

The rheometer is heated to 150° C., the polymer composition is introduced in the rheometer, the gap is set to 1050 μm, excess protruding sample is trimmed, and the gap is then set to 1000 μm. (The axial force control of the rheometer is set to 0 N and be maintained within ±0.1 N of force during the experiment, thereby thermal expansion/contraction of the sample itself is compensated by adjusting the gap in order to avoid overfilling or underfilling in addition to the abovementioned compensation of the tooling.) The rheometer is then allowed to cool to 130° C., at which point the measurement commences with temperature ramped from 130° C. to −10° C. at a constant rate of cooling of 2° C./min (hot to cold temperature ramp). The applied strain amplitude is 0.1%, and the frequency of oscillation is 1 Hz (that is, one cycle per second). The resulting oscillatory stress is recorded.

After this step, the sample temperature is set to 23° C. (temperature is ramped to this setpoint at a rate of 10° C./min), and the sample is allowed to rest for 4.0 hours at 23° C. At the end of this period, the temperature is set to −10° C. (temperature is ramped to this setpoint at a rate of 10° C./min), the sample is equilibrated for 300 seconds at −10° C., and a second oscillatory rheology measurement is conducted (0.1% strain, frequency of oscillation of 1 Hz) while temperature is ramped upward to 130° C. at a constant rate of increase of 2° C./min (cold to hot temperature ramp). The applied strain amplitude is 0.1%, and the frequency of oscillation is 1 Hz (that is, one cycle per second). The resulting oscillatory stress is recorded.

From the first decreasing temperature ramp (hot to cold), the storage modulus G′ and the loss modulus G″ are calculated and recorded from 130° C. until −10° C. in 0.5° C. steps or smaller steps. These values are reported in Pascals (Pa) to the nearest 1 Pa. The storage modulus G′ and the loss modulus G″ are plotted both as y axis in a logarithmic scale against the temperature as x-axis in a linear scale. The single values of the temperature steps are connected to obtain a storage modulus curve G′ and a loss modulus curve G″ verse the temperature. The Cross Over temperature is the temperature where the loss modulus G″ [Pa] and the storage Modus G′ [Pa] become equal and thereby the lines are crossing in the plot. In case more than one Cross Over temperature can be determined in the decreasing temperature ramp (hot to cold), only the highest Cross Over temperature is reported. The Cross Over is reported to the nearest 1° C.

From the second increasing temperature ramp (cold to hot) the storage modulus G′ is calculated and recorded at 37° C., and these values are reported in Pascals (Pa) to the nearest 1 Pa as the “Storage Modulus at 37° C.”.

### Extensional Test Method

The Extensional Test Method is used to determine the Yield Stress and the Toughness for a specimen of a polymer composition. A thin film specimen formed of polymer composition is analyzed with a rotational rheometer fitted with a specialized fixture with counter rotating rollers, and the stress associated with extensional strain imparted is measured and recorded.

### Instrumental Setup

A rotational rheometer (ARES G2, TA Instruments, New Castle, Del., USA, or equivalent) is fitted with a fixture that has counter rotating cylindrical rollers specifically designed for the interrogation of extension deformation of films. An example of a suitable fixture is the Extensional Viscosity Fixture, or EVF (EVF, TA Instruments, or equivalent). The rheometer is further fitted with a forced-convection oven FCO (FCO, TA Instruments, or equivalent) and cooling system (ACS 2, TA Instruments, or equivalent) capable of controlling temperate from at least −50 to 250° C. to a within a tolerance of 0.5° C.

### Specimen Preparation

Approximately 6 g±2 g of the polymer composition is placed in a circular polytetrafluoroethane (PTFE) bowl with a flat bottom (diameter of 60 mm±2 mm) and introduced into a vacuum oven held at 170° C. After 15 minutes at ambient pressure, the pressure is lowered to 10 mbar, and the polymer composition is subsequently held at 170° C. and at 10 mbar for 45 minutes to remove air bubbles from the polymer composition. If 170° C. is insufficient to melt the polymer compositions a temperature 30±10 ° C. above the melting temperature of the polymer material composition is used. The polymer composition is removed from the vacuum oven and allowed to cool to ambient lab conditions (23±2° C.) for 90±30 minutes, at which point the polymer composition is removed from the PTFE bowl and placed between 2 sheets of siliconised paper (such as product number 114918, Mondi Group, Hilm, Austria, or equivalent). A metal shim 500±30 μm in thickness is used in the heated press as a spacer to obtain a film thickness of 500 μm when pressed with a heated press at 90° C. for 60 seconds at a pressure sufficient to form a polymeric film. If 90° C. is insufficient to press a uniform flat film, a temperature approximately 10±5 ° C. below the melting point of the sample material composition such that the sample material composition is in a semi-solid state is used. The film is stored at least 120 hours in the laboratory at 23±2 ° C. prior to testing. From the film individual specimens for measurement are punched with a sample cutter to the final specimen dimensions of 20.0 mm by 10.0 mm by 500 μm.

### Measurement

To secure the specimen film to the cylinders of the EVF, the cylinders are heated to 50° C. for 90±30 s in the forced-convection oven of the rheometer. After opening the oven, the specimen of polymer composition is briefly pressed onto the cylinders of the EVF to secure it to the cylinder surfaces. The specimen is placed with its length perpendicular to the axis of rotation of the cylinders. For polymer compositions, which are very stiff and do not adhere to the cylinder surface, the EVF are heated to 80° C. for 90±30 s in the forced-convection oven of the rheometer. Then a small droplet (0.03±0.01 g) of an auxiliary hotmelt adhesive is applied to each cylinder. The used auxiliary adhesive should exhibit a high stiffness (G′ at 23° C. and 1 Hz of the auxiliary adhesive greater than 10 MPa) to not interfere with the measurement. The specimen of polymer composition is quickly pressed on the auxiliary adhesive on the cylinders of the EVF to fix it to the cylinder surfaces. The specimen is placed perpendicular to the axis of rotation of the cylinders.

The specimen mounted on the EVF is then placed in the forced convection oven of the rheometer for thermal conditioning and is kept isothermal at 37±0.5° C. for 300±10 s. After this time has elapsed, the specimen is mechanically conditioned. To mechanically condition the specimen, the torque transducer is zeroed, and the sample is put under a pre-stretch rate of 0.001 s⁻¹ for 0.30 s and then allowed to relax for 60 s (in this method, all strain is expressed in terms of Hencky strain, also known as “true strain” or “logarithmic strain.”).

The measurement is performed in the FCO oven at 37° C.±0.5° C. The strain rate extension for the measurement is 1 s⁻¹, and the strain at maximum extension is 4.0. After measurement, the specimen is checked for rupturing. If it has ruptured, the location of the break is noted. If the rupture is approximately in the middle between the two cylinders of the EVF, the data collected are deemed acceptable. Otherwise, if the polymeric film break is at or close to the rotating cylinders, the results are discarded, and the measurement performed again on a replicate specimen.

### Analysis

For the extensional stress calculation, a constant volume is assumed. From the raw torque versus angular displacement data recorded by the rheometer, extensional stress (in megapascals, or MPa) versus Hencky strain data are calculated. The data are plotted in semilogarithmic fashion with Hencky strain on the abscissa (linear scale) and extensional stress on the ordinate (logarithmic scale). A linear range is sought in this plot. If a linear range above a strain of 0.3 can be identified and this range can be fit with a positive slope with an R² value of 0.98 or greater, the value of the fitted line at a Hencky strain of zero (that is, the y-intercept), is defined as the Yield Stress, which is reported in MPa to the nearest tenth of MPa. Otherwise, the maximum value of extensional stress recorded during the measurement is reported as the Yield Stress, again reported in MPa to the nearest tenth of MPa.

The extensional stress (MPa) versus Hencky strain data calculated above are again plotted, but this time in linear fashion with Hencky strain on the abscissa (linear axis) and extensional stress on the ordinate (linear axis). The integral of extensional stress with strain (that is, the area under the extensional stress curve as a function of strain) is calculated from a strain of zero to the strain at which the sample ruptured (or, in the case it did not rupture during the measurement, to a strain of 4.0) and is reported as the Toughness, which is reported in units of megajoules per cubic meter, or MJ/m³.

### Misc

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hotmelt adhesive comprising:
at least one low molecular weight metallocene-catalyzed polyolefin having a peak molecular weight below 130,000 g/mol, wherein the peak molecular weight is measured according to the Peak Molecular Weight (Mp) Measurement Method;
at least one high molecular weight polyolefin having a peak molecular weight of from about 130,000 g/mol to about 700,000 g/mol; and
at least one tackifier;
wherein the hotmelt adhesive comprises less than 10% by weight of mineral oil.

2. The hotmelt adhesive according to claim 1, wherein the low molecular weight metallocene-catalyzed polyolefin is a propylene-based polymer, in particular a propylene-ethylene copolymer.

3. The hotmelt adhesive according to claim 1, wherein the hotmelt adhesive comprises:
a first low molecular weight metallocene-catalyzed polyolefin, which is a metallocene-catalyzed propylene-ethylene copolymer having a Heat of Crystallization below 20 J/g, as measured by the Heat of Crystallization Test Method;
a second low molecular weight metallocene-catalyzed polyolefin, which is a metallocene-catalyzed propylene-ethylene copolymer having a Heat of Crystallization above 20 J/g.

4. The hotmelt adhesive according to claim 3, wherein the first low molecular weight metallocene propylene-ethylene copolymer has a Heat of Crystallization in the range of from about 5 J/g to about 15 J/g, and the second low molecular weight metallocene-catalyzed propylene-ethylene copolymer has a Heat of Crystallization in the range of from about 25 J/g to about 45 J/g.

5. The hotmelt adhesive according to claim 4, wherein the first low molecular weight metallocene propylene-ethylene copolymer has a viscosity at 170° C. below 500 mPa.s, as measured by the Viscosity Test Method.

6. The hotmelt adhesive according to claim 1, wherein the hotmelt adhesive comprises a single low molecular weight metallocene-catalyzed polyolefin, and the single low molecular weight metallocene-catalyzed polyolefin has a Heat of Crystallization in the range of about 20 J/g to about 30 J/g and a peak molecular weight between about 25,000 and about 35,000 g/mol.

7. The hotmelt adhesive according to claim 1, wherein the high molecular weight polyolefin is a metallocene-catalyzed propylene-based polymer.

8. The hotmelt adhesive according to claim 1, wherein the high molecular weight polyolefin has a peak molecular weight ranging from about 130,000 g/mol to about 410,000 g/mol.

9. The hotmelt adhesive according to claim 8, wherein the high molecular weight polyolefin has a peak molecular weight ranging from about 150,000 g/mol to about 360,000 g/mol.

10. The hotmelt adhesive according to claim 1, wherein the hotmelt adhesive comprises by weight:
from about 10% to about 70% of the low molecular weight metallocene-catalyzed polyolefin(s),
from about 1% to about 20% of the high molecular weight polyolefin(s), and
from about 10% to about 60% of the tackifier(s).

11. The hotmelt adhesive according to claim 10, wherein the hotmelt adhesive comprises by weight:
from about 30% to about 60% of the low molecular weight metallocene-catalyzed polyolefin (s),
from about 5% to about 15% of the high molecular weight polyolefin(s),
from about 15% to about 50% of the at least one tackifier.

12. The hotmelt adhesive according to claim 11, wherein the hotmelt adhesive comprises more than 30% of the at least one tackifier.

13. The hotmelt adhesive according to claim 1, wherein the hotmelt adhesive comprises less than 5% by weight of mineral oil.

14. The hotmelt adhesive according to claim 13, wherein the hotmelt adhesive comprises less than 1% by weight of mineral oil.

15. The hotmelt adhesive according to claim 1, wherein the viscosity of the hotmelt adhesive at 170° C. is in the range from about 1,000 mPa·s to about 7,000 mPa·s, as measured according to the Viscosity Test Method.

16. The hotmelt adhesive according to claim 1, wherein the hotmelt adhesive has a Toughness of at least 11 MJ/m³, as measured by the Extension Test Method.

17. The hotmelt adhesive according to claim 16, wherein the hotmelt adhesive has a Toughness of at least 25 MJ/m³, as measured by the Extension Test Method.

18. The hotmelt adhesive according to claim 1, wherein the hotmelt adhesive has a least one property selected from the following:
a storage modulus (G′) at 37° C. larger than 3.0 MPa, as measured in the cold to hot temperature ramp by the Oscillatory Rheometry Test Method;
a Yield Stress at 37° C. larger than 0.7 MPa as measured by Extension Test Method; and
a Cross Over temperature below 75° C., as measured in the hot to cold temperature ramp according to the Oscillatory Rheometry Test Method.

19. An absorbent article comprising a topsheet, a backsheet, an absorbent core between the topsheet and the backsheet, wherein the absorbent core comprises a core wrap having a top layer and a bottom layer and an absorbent material comprising superabsorbent particles, wherein the hotmelt adhesive according to claim 1 forms a core wrap bond between the top layer and the bottom layer of the core wrap.

20. An absorbent article comprising a topsheet, a backsheet, an absorbent core between the topsheet and the backsheet, a distribution layer between the absorbent core and the topsheet, wherein the absorbent core comprises a core wrap having a top layer and a bottom layer and an absorbent material comprising superabsorbent particles, wherein the hotmelt adhesive according to claim 1 forms a patch-to-core bond between the distribution layer and the top layer of the core wrap.
